(19) United States
(12) Patent Application Publication
(10) Pub. No.: US 2026/0345143 A1
(43) Pub. Date: Nov. 5, 2026

(54) ACTUATOR ASSEMBLIES, RELATED SYSTEMS, AND RELATED METHODS

(71) Applicant: Boston Scientific Scimed, Inc., Maple Grove, MN (US)

(72) Inventors: Alexander COHEN, Austin, TX (US); John MIMS, Austin, TX (US); Jason N. MEHTA, Austin, TX (US); Timothy Noah FOSTER-FRAU, Austin, TX (US)

(73) Assignee: Boston Scientific Scimed, Inc., Maple Grove, MN (US)

(21) Appl. No.: 19/661,785

(22) Filed: Apr. 29, 2026

Related U.S. Application Data
(60) Provisional application No. 63/798,017, filed on May 1, 2025.

(51) Int. Cl. A61B 17/00; A61B 1/018

(52) U.S. Cl. CPC A61B 17/0057; A61B 1/018; A61B 17/00234; A61B 2017/00296; A61B 2017/00367; A61B 2017/00477; A61B 2017/00584; A61B 2017/00623

(57) ABSTRACT

A medical instrument may include a shaft. The shaft may include a proximal portion and a distal portion. The medical instrument may include an end effector disposed at the distal portion of the shaft. The medical instrument may include an actuator assembly disposed at the proximal portion of the shaft. The actuator assembly may include a body and an actuator movable relative to the body. The medical instrument may include a locking mechanism disposed on the body. The locking mechanism may include a locking arm and a locking portion. The locking portion may include a channel configured to engage the locking arm. The locking mechanism may be configured to selectively lock the actuator in at least one configuration. The medical instrument may include an actuation wire extending from the actuator assembly through the shaft to the end effector.

### CROSS-REFERENCE TO RELATED APPLICATIONS

This application claims the benefit of priority to U.S. Provisional Application No. 63/798,017, filed on May 1, 2025, which is incorporated by reference herein in its entirety.

### FIELD OF INVENTION

Various aspects of this disclosure relate generally to systems, devices, and methods of medical instrument actuator assemblies, related systems, and related methods, for example, to control end effectors. In particular, aspects of this disclosure relate to medical instruments with locking and actuation mechanisms that secure or actuate an end effector during a medical procedure.

### BACKGROUND

Medical procedures often involve manipulating tissue via one or more medical instruments. For instance, endoscopic procedures utilize medical instruments to access and treat tissue within the gastrointestinal tract or other internal body cavities. In such procedures, an endoscope with a working channel may be used to deliver medical instruments like graspers or clips to a target treatment site. These instruments may be used to grasp, cut, or otherwise manipulate tissue. However, challenges can arise during these procedures, particularly when complex coordination is required between the physician and assisting staff. For example, when applying advanced closure techniques using clips, a choreographed effort is required among multiple surgical team members, which may introduce communication failures and complications. Additionally, recent trends in hospital staffing, combined with the increasing complexity of therapeutic endoscopy, have emphasized a need for physician-centric tools that reduce reliance on support staff.

The devices, systems, and methods of this disclosure may help to rectify one or more of the deficiencies described above or address other aspects of the art.

### SUMMARY

Each of the aspects disclosed herein may include one or more of the features described in connection with any of the other disclosed aspects.

This disclosure includes a medical instrument that may include a shaft. The shaft may include a proximal portion and a distal portion. The medical instrument may include an end effector disposed at the distal portion of the shaft. The medical instrument may include an actuator assembly disposed at the proximal portion of the shaft. The actuator assembly may include a body and an actuator movable relative to the body. The medical instrument may include a locking mechanism disposed on the body. The locking mechanism may include a locking arm and a locking portion. The locking portion may include a channel configured to engage the locking arm. The locking mechanism may be configured to selectively lock the actuator in at least one configuration. The medical instrument may include an actuation wire extending from the actuator assembly through the shaft to the end effector. Movement of the actuator relative to the body may cause corresponding movement of the actuation wire to actuate the end effector.

The medical instrument may include any of the following features. The locking arm may be pivotally coupled to the body via a pin. The locking portion may be disposed on the actuator. The locking arm may include a proximal portion pivotally coupled to the pin and a distal portion. The distal portion may include a protrusion configured to movably engage with the locking portion of the actuator. The channel may include a first ramp, a second ramp, and a locking slot disposed between the first ramp and the second ramp. The locking slot may include a distalmost portion that may be distal of a proximalmost portion of the first ramp or a proximalmost portion of the second ramp. The channel may be chevron-shaped and may include the first ramp, the second ramp, and the locking slot. The protrusion of the locking arm may translate through the channel as the actuator may be actuated. When the protrusion of the locking arm may be positioned at a distalmost position of the chevron-shaped channel, the end effector may be in a closed position. When the protrusion of the locking arm may be positioned in the locking slot, the end effector may be in an open position. The medical instrument may include a biasing element disposed within the body and configured to bias the actuator proximally relative to the body. The shaft may include a stretchable portion proximal to the end effector. The stretchable portion may be configured to stretch during actuation of the actuator. Stretching of the stretchable portion may apply a distal force configured to deploy the end effector. The end effector may include a clip. The first ramp and the second ramp each may include an angled portion and a straight portion. The angled portions may be angled relative to a central longitudinal axis of the medical instrument. The straight portions may be parallel to the central longitudinal axis of the medical instrument. The first ramp and the second ramp may each be a same depth. The locking slot may be of greater depth than the first ramp or the second ramp. A distalmost portion of the first ramp and a distalmost portion of the second ramp may be distal to a proximalmost portion of the locking slot. The end effector may include a grasper.

This disclosure also includes a medical system that may include an insertion device. The insertion device may include a handle, a port, and a shaft extending distally from the handle. The shaft may define a working channel in communication with the port. The medical system may include a medical instrument configured to be coupled to the port and be extended through the working channel. The medical instrument may include a shaft. The shaft may include a proximal portion and a distal portion. The medical instrument may include an end effector disposed at the distal portion of the shaft. The medical instrument may include an actuator assembly disposed at the proximal portion of the shaft. The actuator assembly may include a body and an actuator movable relative to the body. The medical instrument may include a locking mechanism disposed on the body and configured to selectively lock the actuator in at least one configuration. The locking mechanism may include a locking portion with a locking slot. The medical instrument may include a biasing element disposed within the body and configured to bias the actuator proximally relative to the body. The medical instrument may include an actuation wire extending from the actuator assembly through the shaft to the end effector. Movement of the actuator relative to the body may cause corresponding movement of the actuation wire to actuate the end effector. A force exerted by the biasing element may oppose actuation of the actuator.

The medical system may include any of the following features. The locking mechanism may include a locking arm with a protrusion configured to engage with the locking slot. A first configuration of the medical system may include the protrusion positioned at a distalmost position of the locking portion. The end effector may be in a closed position in the first configuration. The biasing element may bias the protrusion against the distalmost position of the locking portion. A second configuration of the medical system may include the protrusion positioned in the locking slot. The end effector may be in an open position in the second configuration. The biasing element may bias the protrusion against the distalmost position of the locking slot. The locking portion may include a first ramp and a second ramp. The first ramp and the second ramp each may include an angled portion and a straight portion. The angled portions may be angled relative to a central longitudinal axis of the medical instrument. The straight portions may be parallel to the central longitudinal axis of the medical instrument.

This disclosure also includes a medical instrument that may include a shaft. The shaft may include a proximal portion and a distal portion. The medical instrument may include an end effector disposed at the distal portion of the shaft. The medical instrument may include an actuator assembly disposed at the proximal portion of the shaft. The actuator assembly may include a body and an actuator movable relative to the body. The actuator assembly may include a locking mechanism. The locking mechanism may include a first ramp disposed on the actuator and may include a straight portion and an angled portion. The locking mechanism may include a second ramp disposed on the actuator and may include a straight portion and an angled portion. The locking mechanism may include a locking slot disposed on the actuator between the first ramp and the second ramp. The locking mechanism may include a locking arm pivotally coupled to the body via a pin. The locking arm may include a protrusion configured to interact with the first ramp, the second ramp, or the locking slot upon actuation of the actuator. The medical instrument may include a biasing element disposed within the body and configured to bias the actuator proximally relative to the body. The medical instrument may include an actuation wire extending from the actuator assembly through the shaft to the end effector. The biasing element may apply a proximally-directed force to the actuator to maintain the end effector in a first configuration or a second configuration. Actuation of the actuator against the proximally-directed force of the biasing element may cause the actuation wire to transition the end effector from the first configuration to the second configuration.

The medical instrument may include any of the following features. The first ramp, the second ramp, and the locking slot may form a chevron-shaped channel. The protrusion of the locking arm may translate through the channel as the actuator may be actuated.

### BRIEF DESCRIPTION OF FIGURES

FIG. 1 illustrates an exemplary medical system including a medical instrument and an end effector extending from a distal end of a medical device, according to aspects of the disclosure.

FIG. 2 illustrates a side view of a medical instrument, according to an embodiment, according to aspects of the disclosure.

FIG. 3A illustrates a partially transparent view of a medical instrument actuator assembly in a first configuration, according to aspects of this disclosure.

FIG. 3B illustrates a partially transparent view of the medical instrument actuator assembly in a second configuration, according to aspects of this disclosure.

### DETAILED DESCRIPTION

Aspects of this disclosure are described in greater detail below. The terms and definitions provided herein control, if in conflict with terms and/or definitions incorporated by reference.

The terms "proximal" and "distal" are used herein to refer to the relative positions of the components of exemplary medical devices. As used herein, "proximal" refers to a position relatively closer to the exterior of the body or closer to an operator using the medical device. In contrast, "distal" refers to a position relatively further away from the operator using the medical device, or closer to the interior of the body. Several drawings include arrows labeled “P” and “D,” indicating proximal and distal directions, respectively. As used herein, the terms "comprises," "comprising," "including," "includes," "having," "has," or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term "exemplary" is used in the sense of "example," rather than "ideal." Further, relative terms such as, for example, "about," "substantially," "approximately," etc., are used to indicate a possible variation of ±10 % in a stated numeric value or range. As used herein, the phrase "based on" is understood to be equivalent to the phrase "based at least on," unless indicated otherwise. The term "or" is used disjunctively, such that "at least one of A or B" includes, (A), (B), (A and B), (A or B), (B or A), (A and A), (B and B), etc. It will also be understood that, although the terms first, second, third, etc. are, in some instances, used herein to describe various elements, these elements should not be limited by these terms. These terms are only used to distinguish one element from another.

Although scopes and endoscopes are referenced herein for illustration purposes, it will be appreciated that the disclosure encompasses any suitable medical device configured to allow an operator to access and view internal body anatomy of a subject and/or to deliver medical instruments. As such, aspects of this disclosure include insertion or medical devices that may be used to deliver one or more of, for example, biopsy forceps, graspers, baskets, snares, probes, scissors, capture/retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography ("EUS") scopes, colonoscopes, ureteroscopes, bronchoscopes, laparoscopes, cystoscopes, aspiration scopes, sheaths, catheters, or any other suitable delivery device may be used in connection with the features described herein.

This disclosure describes actuation mechanisms for medical instruments. Features of the actuation mechanisms described herein may help to improve control of components involved with manipulating tissue at a treatment site by a single operator, such a physician. Enhanced control of tissue at the treatment site may help to reduce duration of medical procedures (e.g., ESD), increase efficiency and/or safety of medical procedures, for example by manipulating tissue to as part of a medical procedure, among other aspects.

Recent trends in hospital staffing and the increasing complexity of therapeutic endoscopy have emphasized a need for more physician-centric tools. These tools may aim to provide physicians with greater control over medical procedures, potentially reducing reliance on support staff. In some cases, medical instruments such as clips or graspers with physician-controllable states and deployment mechanisms may allow physicians to maintain control over a greater share of the steps of a medical procedure. This physician-centric approach may help streamline procedures by reducing the training requirements for technicians and support staff and minimizing communication-related issues that can arise during complex procedures involving multiple team members. A physician-centered design may reduce the need for carefully coordinated efforts among multiple team members to perform one or more portions of a medical procedure.

This disclosure describes medical instruments with actuator assemblies for use with insertion devices. Features of the actuator assemblies described herein may improve physician control of tissue manipulating instruments and end effectors. For example, the systems, devices, and methods described herein may provide locking and actuation mechanisms that secure the position of an end effector during a medical procedure, while also allowing for controlled actuation when needed. Enhanced control of medical instruments at the treatment site may help to reduce a duration of a medical procedure, increase efficiency and/or safety of medical procedures, for example by enabling precise manipulation of tissue to improve access to the treatment site, among other aspects.

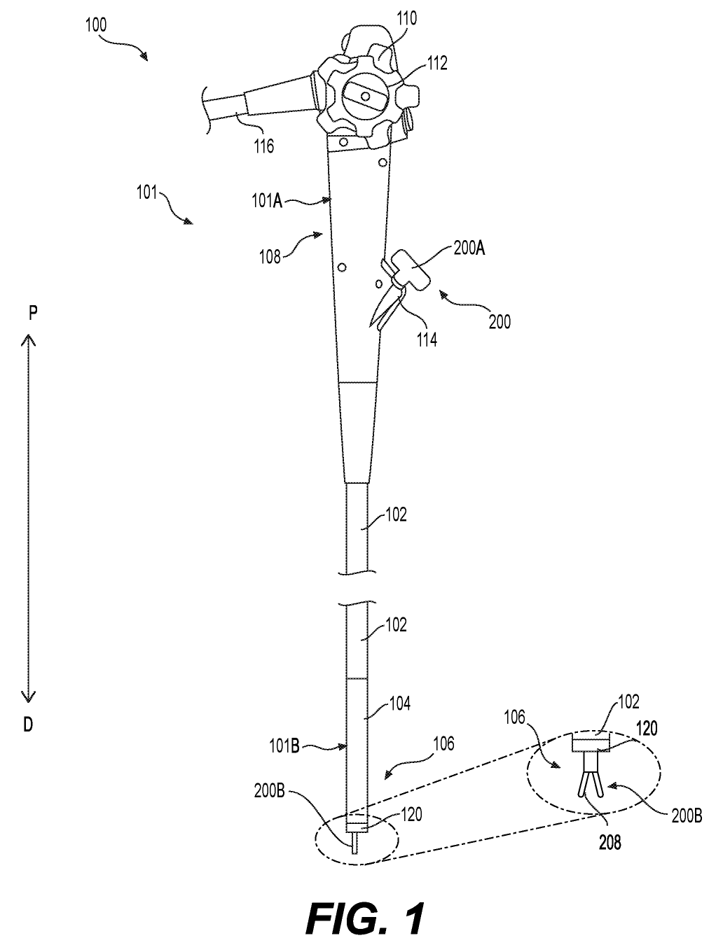

Referring to FIG. 1, a medical system 100 may include an insertion device 101 and a medical instrument 200. Insertion device 101 may include a handle 108 and a shaft 102, with shaft 102 extending distally from handle 108. Shaft 102 may include an articulation section 104 and a distal tip or end 106. A distal opening of shaft 102 may be in communication with a working channel extending through shaft 102. As shown in FIG. 1, handle 108 may also include a port 114, which may be in communication with the working channel of insertion device 101. Handle 108 may include one or more actuators, for example, a first control member 110 and a second control member 112 (e.g., knobs or levers), for controlling articulation section 104 of shaft 102. For example, control members 110 and 112 may control movement of articulation section 104 of shaft 102 in multiple directions, for example, movement along different planes. Handle 108 may be coupled to an umbilicus 116. Umbilicus 116 may extend from handle 108 to capital equipment, such as electronic controller(s), one or more displays, one or more user interfaces, and/or source(s) of air, water, and/or suction. Furthermore, medical instrument 200 includes a proximal portion 200A and a distal portion 200B. As discussed in detail below, proximal portion 200A may include one or more actuators or one or more features that control various aspects of distal portion 200B. For example, a user (e.g., a physician) may actuate one or more control members on proximal portion 200A (e.g., first control member 110) to articulate or otherwise control the position of distal portion 200B as part of a medical procedure.

Distal portion 200B of medical instrument 200 can be maneuvered through working channel 118 and extended beyond distal end 106 of shaft 102, allowing for controlled advancement and retraction during the medical procedure. When actuated, end effector 208 can open and close in response to physician actuation of actuator assembly 202, enabling precise grasping, clipping, or manipulation of target tissue

In some aspects, insertion device 101 may receive medical instrument 200, for example, through the working channel via port 114. As mentioned above, the working channel is in communication with port 114 (FIG. 1). As such, medical instrument 200 may be inserted through port 114, through the working channel in shaft 102, and out of distal end 106 to be delivered to a treatment site. Medical instrument 200 may include an end effector 208, such as a clip or grasper, and a portion of medical instrument 202 (e.g., including end effector 208) may be extended through port 114 and through a working channel of insertion device 101.

In some aspects, medical instrument 200 may incorporate various types of end effectors suitable for different medical procedures. While clips and graspers are mentioned, other end effectors may also be utilized, including but not limited to scissors, snares, baskets, forceps, needle holders, biopsy devices, electrosurgical tools, or any other appropriate surgical or diagnostic end effector.

Referring back to the insertion device 101, articulation section 104 may allow movement or positioning of distal portion of shaft 102 (e.g., distal end 106) and shaft 102 (e.g., movement in a radial direction). For example, articulation section 104 may be useful to allow flexibility at distal end 106, such that end effector 208 of medical instrument 200 may be positioned and articulated at the treatment site.

In some aspects, shaft 102 also defines one or more channels therethrough, which may be in fluid communication with one or more lumens defined by handle 108. Handle 108 may also include one or more ports in fluid communication with the channels of shaft 102. The port(s) may be connected to various sources to provide different functionalities through the port(s), the channel(s) of shaft 102, and distal end 106. Alternatively or additionally, handle 108 may be connected to one or more external sources via umbilicus 116. Handle 108 may include one or more features (e.g., valves, buttons, etc.) that allow the user to control or adjust the operation of various functions during a medical procedure. In other aspects, although not shown, medical system 100 may include one or more auxiliary tubes, for example, external to shaft 102 that connect distal end 106.

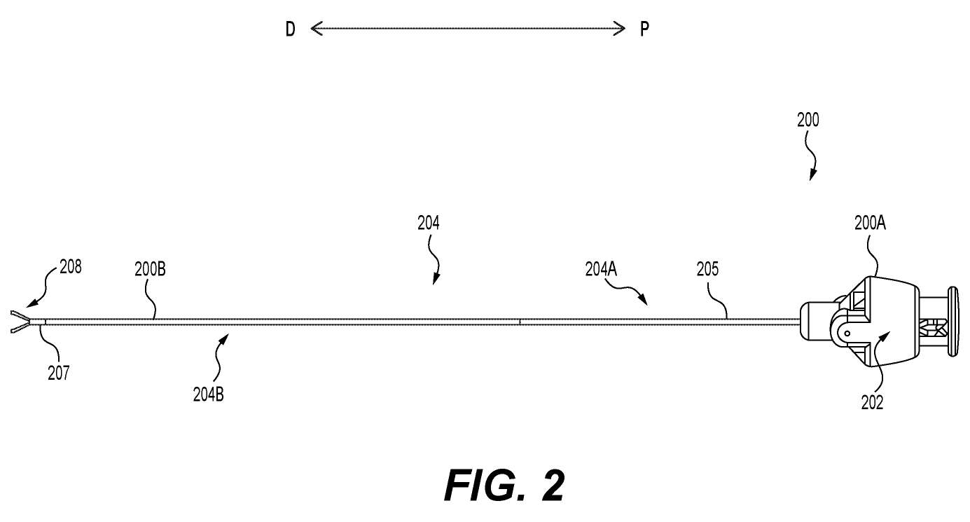

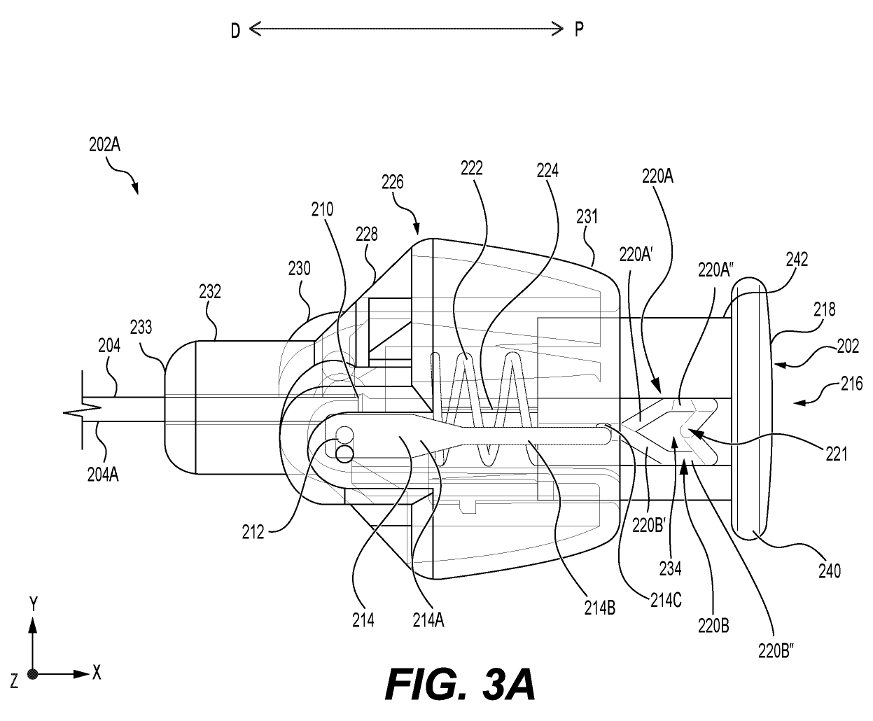

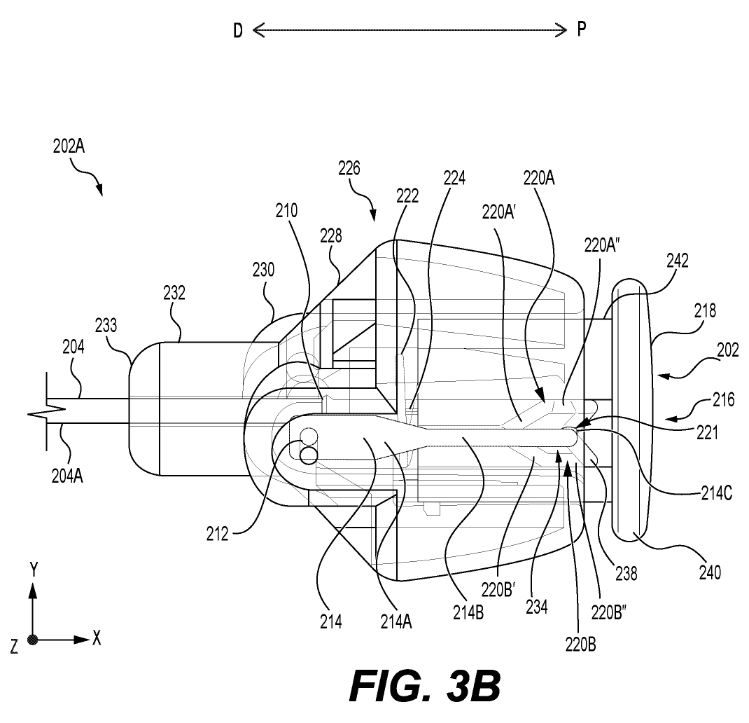

FIG. 2 illustrates additional aspects of medical instrument 200. As mentioned, medical instrument 200 includes proximal portion 200A and distal portion 200B, with distal portion 200B including end effector 208. An actuator assembly 202 may be positioned at a proximal portion 200A of medical instrument 200. Actuator assembly 202 may be configured to control operation of end effector 208. Actuator assembly 202 is depicted and described as a button in FIGS. 2, 3A, and 3B, but one of ordinary skill in the art will appreciate that other actuation mechanisms may be utilized without departing from the scope of the disclosure.

Medical instrument 200 also includes a shaft 204, for example, extending between actuator assembly 202 and end effector 208. Shaft 204 may extend distally from the actuator assembly 202. Shaft 204 may include a proximal portion 204A and a distal portion 204B. Proximal portion 204A may be coupled to actuator assembly 202, and distal portion 204 may be coupled to end effector 208 (e.g., a clip or grasper). Shaft 204 may be flexible, allowing the medical instrument 200 to navigate through the working channel of insertion device 101. One or more actuation wires 224 (FIGS. 3A and 3B) may extend from actuator assembly 202 along or within shaft 204 to end effector 208 to transmit mechanical force from the actuator assembly 202 to cause actuation of end effector 208.

In some aspects, proximal portion 204A of shaft 204 may incorporate a stretchable portion 205, which may be useful when end effector 208 is a deployable clip. Stretchable portion 205 may allow the physician to apply force to end effector 208, in addition to force supplied by actuation wire 224, which transmits force along shaft 204 to end effector 208. When actuation wire 224 is moved or urged proximally, it may cause a proximal end of a clip (e.g., end effector 208) to interact with a distalmost end of shaft 207, which separates the clip for release. This interaction between the proximal end of a clip and the distalmost end 207 of the shaft 204 is primarily relevant for clip deployment, and stretchable portion 205 may be omitted in configurations of medical instrument 200 utilizing a grasper or other non-deployable instruments as end effector 208.

Medical instrument 200 may be configured to bend in conjunction with articulation section 104 of insertion device 101. The physician may use actuator assembly 202 to control end effector 208, while simultaneously manipulating articulation section 104 of insertion device 101 using first control member 110 or second control member 112.

FIG. 3A illustrates a partially transparent view of an actuator assembly 202 in a first configuration, according to aspects of this disclosure. Actuator assembly 202 may include a body 226, which may enclose the various mechanisms of actuator assembly 202, and a button 216. Body 226 may be ergonomically grasped by a physician during a medical procedure. For example, a physician may place the fingers of one hand around body 226, while a thumb rests upon a proximalmost surface 218 of button 216. In other aspects, body 226 may be secured within port 114 (FIG. 1), and the physician may only rest a thumb on proximalmost surface 218 of button 216 Body 226 may include a distal portion 232, which may slope distally outward (e.g., away from a central longitudinal axis of actuator assembly 202). Distal portion 232 may decrease in size (e.g., diameter) in a distal direction towards shaft 204. Distal portion 232 may terminate proximally at an intermediate portion 228, which may be stepped or sloped (e.g., sloped radially inward in the distal downwards towards the central longitudinal axis of actuator assembly 202). Intermediate portion 228 may terminate distally at rounded portion 230. Rounded portion 230 may define a curved portion of actuator body 226 between intermediate portion 228 and distal portion 232. Distal portion 232 may extend distally to a distalmost face 233. Distalmost face 233 may include an opening (not shown) to receive or otherwise be coupled to proximal portion 204A of shaft 204.

Distalmost face 233 may abut port 114 when medical instrument 200 is positioned within insertion device 101. Body 226 may thus include a variety of portions (e.g., stepped intermediate portion 228, rounded portion 230, proximal portion 231, and distal portion 232) configured to be easily and ergonomically grasped and controlled by a physician with a single hand. This configuration may allow a physician to control end effector 208 with one hand, while using another hand for another task.

Button 216 may be affixed to, contiguously formed with, or otherwise include a button shaft 242. Button shaft 242 may extend distally through a proximal opening of body 226. Button 216 may include a button ring 240 extending circumferentially outward from button shaft 242 and the central longitudinal axis of medical instrument 200. Button shaft 242 may translate proximally or distally through the opening of body 226 based on actuation of button 216 by a physician.

Button shaft 242 may include a locking portion 234, which may include one or more cut-away, depressed, removed, or otherwise indented portions which may define one or more pathways or tracks. Additionally, actuator assembly 202 may include a locking arm 214, and locking arm 214 includes a protrusion 214C. As discussed in detail below, locking arm 214 may be movable relative to and interact with locking portion 234. Locking portion 234 may help to allow a physician to lock end effector 208 in various configurations (e.g., open or closed), for example, by manipulating button 216 with a single hand (or one or more fingers or thumbs).

Actuator assembly 202 may include a biasing element 222. Biasing element 222 may be positioned within a portion of actuator assembly 202. In some aspects, a distal end of biasing element 222 may be affixed or otherwise positioned distal to an inner portion of body 226. Additionally or alternatively, a proximal end of biasing element 222 may be affixed or otherwise positioned adjacent to or proximal to a distal end or an interior portion of button 216. Biasing element 222 may bias (e.g., apply a force upon) button 216 proximally, such that a distally-directed force applied to button 216 must oppose and overcome the proximally-directed force provided by biasing element 222. While biasing element 222 is depicted as a spring in FIGS. 3A and 3B, other biasing mechanisms are within the scope of the disclosure. For example, biasing element 222 may comprise magnets of opposing polarity affixed to body 226 and an inner surface of button 216.

Actuator assembly 202 may include or otherwise be coupled to an actuation wire 224. Actuation wire 224 may be affixed proximally to a distal end or an interior portion of button 216, extending distally through biasing element 222 and body 226 to a shaft interface 210. Shaft interface 210 may be understood as the proximalmost portion of shaft 204 and may be affixed to an interior portion of button 216. Actuation wire 224 may extend distally through a lumen of shaft interface 210 and through a lumen of shaft 204 to a proximal portion of end effector 208 (FIG. 2).

Referring back to locking arm 214, locking arm 214 may include a proximal portion 214A, which may include a pin 212. Locking arm 214 may also include a distal portion 214B, which may include a protrusion 214C. Protrusion 214C may extend radially inward relative to distal portion 214B. Locking arm 214 may be rotatable or pivotable about pin 212, for example, as protrusion 214C interacts with portions of the locking portion 234.

Locking portion 234 may include a first ramp 220A, a second ramp 220B, and a locking slot 221. First ramp 220A may include a distal angled portion 220A’ and an intermediate straight portion 220A’’. Second ramp 220B may also include a distal angled portion 220B’ and an intermediate straight portion 220B’’. First ramp 220A and second ramp 220B may have a same Z-depth (e.g., extending into/out of the page of FIGS. 3A and 3B) relative to the central longitudinal axis of actuator assembly 202. In some aspects, locking slot 221 may have a same Z-depth as ramps 220A/220B, but in other aspects, locking slot 221 may have a greater Z-depth as compared to ramps 220A/220B. For example, locking slot 221 may be closer to the central longitudinal axis (e.g., further from the page of FIGS. 3A and 3B) of actuator assembly 202 as compared to ramps 220A/220B.

A user may actuate button 216, which causes button 216 to translate proximally relative to body 226 and locking arm 214. As button 216 is translating proximally, protrusion 214C may slide along first ramp 220A, as first ramp 220A translates (e.g., with button 216) proximally relative to protrusion 214C. The slope of first ramp 220A (or second ramp 220B) imparts a torque upon locking arm 214 via protrusion 214C, which causes locking arm 214 to rotate about pin 212.

After protrusion 214C reaches a distal end of first ramp 220A (or second ramp 220B), protrusion 214C may then slide into locking slot 221, which is proximal of the distalmost end of first ramp 220A and the distamost end of second ramp 220B. Together, locking portion 234 (e.g., first ramp 220A, second ramp 220B, and locking slot 221) may form a chevron-shaped channel through which protrusion 214C may slide along as button 216 is actuated.

In the first configuration shown in FIG. 3A, biasing element 222 may bias button 216 proximally. In this first configuration, protrusion 214C is positioned at a distalmost position of locking portion 234. In this first (e.g., neutral) position, an end effector, such as a clip or grasper may be in a closed position. The first configuration may be used to deliver distal portion 204B and end effector 208 through shaft 102 of insertion device 101, for example, to a position such that end effector 208 is distal to distalmost end of insertion device 120, as shown in FIG. 1. When a user actuates (e.g., depresses) button 216, protrusion 214C may slide and pivot via pin 212 relative to one of first ramp 220A or second ramp 220B. Actuation of button 216 also imparts a force upon actuation wire 224, which moves with button 216. Movement of actuation wire 224 relative to shaft 204 may cause actuation of end effector 208.

After sliding along one of first ramp 220A or second ramp 220B, protrusion 214C may be positioned in locking slot 221 (shown in FIG. 3B). Protrusion 214C may be held in place in locking slot 221 by biasing element 222 applying proximally-directed force to button 216 (e.g., including locking slot 221) relative to locking arm 214 and protrusion 214C. The chevron-shaped configuration of locking portion 234 helps to ensure that protrusion 214C is locked in locking slot 221. This second configuration, shown in FIG. 3B, may lock end effector 208 in an open configuration. For example, the jaws of a grasper end effector 208 may be held open in the second configuration.

A user may actuate button 216 again to transition medical instrument 200 from the second configuration back to the first configuration. When the user applies a distally-directed force to button 216, the force overcomes the proximally-directed force of biasing element 222. This causes button 216 and button shaft 242 to translate distally relative to body 226.

As button shaft 242 moves distally, locking portion 234 also translates distally relative to locking arm 214. This translation causes protrusion 214C to disengage from locking slot 221. Protrusion 214C then slides along second ramp 220B (or first ramp 220A) as locking portion 234 continues to move distally. The slope of second ramp 220B (or first ramp 220A) imparts a torque on locking arm 214 via protrusion 214C, causing locking arm 214 to rotate about pin 212.

When the user releases button 216, biasing element 222 applies a proximally-directed force to button 216. This causes button 216, button shaft 242, and locking portion 234 to translate proximally relative to body 226 and locking arm 214. Protrusion 214C slides along the distal straight portion of second ramp 220B until it reaches the distalmost position of locking portion 234, returning to the first configuration shown in FIG. 3A.

The distal translation of button 216 during this transition also moves actuation wire 224 distally relative to shaft 204. This distal movement of actuation wire 224 causes end effector 208 to return to its closed (e.g., first) configuration. For example, if end effector 208 is a grasper, its jaws would close as medical instrument 200 returns to the first configuration.

A user may actuate button 216 as many times as desired to repeatedly actuate end effector 208 and actuator assembly 202 between the first (e.g., closed jaws) and second configurations (e.g., open jaws). End effector 208 may remain in a given configuration (e.g., first or second) until the user actuates button 216 again due to the force exerted by biasing element 222. The user may wish to use both configurations repeatedly throughout a medical procedure as needed. In aspects utilizing a clip for end effector 208, the repeated actuation of button may correspond to repeated deployment of clips at the target treatment site. The ergonomic arrangement of actuator assembly 202 and button 216 may enable rapid, simplified, and precise control of end effector 208, thereby reducing the need for support staff when operating medical instrument 200.

A user may actuate button 216 as many times as desired to repeatedly actuate end effector 208 and actuator assembly 202 between the first (e.g., closed jaws) and second configurations (e.g., open jaws). End effector 208 may remain in a given configuration (e.g., first or second) until the user actuates button 216 again. The user may wish to use both configurations repeatedly throughout a medical procedure as needed. In aspects utilizing a clip for end effector 208, the repeated actuation of button may correspond to repeated deployment of at the target treatment site. The ergonomic arrangement of actuator assembly 202 and button 216 may enable rapid, simplified, and precise control of end effector 208, thereby reducing the need for support staff when operating medical instrument 200.

While principles of the disclosure are described herein with reference to illustrative aspects for particular medical procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

We claim:

1. A medical instrument, comprising:
 a shaft comprising a proximal portion and a distal portion;
an end effector disposed at the distal portion of the shaft;
an actuator assembly disposed at the proximal portion of the shaft, the actuator assembly including:
a body;
an actuator movable relative to the body; and
a locking mechanism disposed on the body, the locking mechanism including:
a locking arm; and
a locking portion comprising a channel configured to engage the locking arm, wherein the locking mechanism is configured to selectively lock the actuator in at least one configuration; and
an actuation wire extending from the actuator assembly through the shaft to the end effector, wherein movement of the actuator relative to the body causes corresponding movement of the actuation wire to actuate the end effector.

2. The medical instrument of claim 1, wherein the locking arm is pivotally coupled to the body via a pin, wherein the locking portion is disposed on the actuator.

3. The medical instrument of claim 2, wherein the locking arm includes a proximal portion pivotally coupled to the pin and a distal portion comprising a protrusion configured to movably engage with the locking portion of the actuator.

4. The medical instrument of claim 3, wherein the channel further comprises:
a first ramp;
a second ramp; and
a locking slot disposed between the first ramp and the second ramp, the locking slot including a distalmost portion that is distal of (i) a proximalmost portion of the first ramp or (ii) a proximalmost portion of the second ramp.

5. The medical instrument of claim 4, wherein the channel is chevron-shaped and includes the first ramp, the second ramp, and the locking slot, wherein the protrusion of the locking arm translates through the channel as the actuator is actuated.

6. The medical instrument of claim 5, wherein, when the protrusion of the locking arm is positioned at a distalmost position of the chevron-shaped channel, the end effector is in a closed position.

7. The medical instrument of claim 6, wherein, when the protrusion of the locking arm is positioned in the locking slot, the end effector is in an open position.

8. The medical instrument of claim 1, further comprising a biasing element disposed within the body and configured to bias the actuator proximally relative to the body.

9. The medical instrument of claim 1, wherein the shaft comprises a stretchable portion proximal to the end effector.

10. The medical instrument of claim 9, wherein the stretchable portion is configured to stretch during actuation of the actuator, wherein stretching of the stretchable portion applies a distal force configured to deploy the end effector.

11. The medical instrument of claim 9, wherein the end effector comprises a clip.

12. The medical instrument of claim 5, wherein the first ramp and the second ramp each include an angled portion and a straight portion, wherein the angled portions are angled relative to a central longitudinal axis of the medical instrument, wherein the straight portions are parallel to the central longitudinal axis of the medical instrument.

13. The medical instrument of claim 5, wherein the first ramp and the second ramp are each a same depth, wherein the locking slot is of greater depth than the first ramp or the second ramp.

14. The medical instrument of claim 5, wherein a distalmost portion of the first ramp and a distalmost portion of the second ramp are distal to a proximalmost portion of the locking slot.

15. The medical instrument of claim 5, wherein the end effector comprises a grasper.

16. A medical system, comprising:
an insertion device including a handle, a port, and a shaft extending distally from the handle, the shaft defining a working channel in communication with the port; and
a medical instrument configured to be coupled to the port and be extended through the working channel, the medical instrument including:
a shaft comprising a proximal portion and a distal portion;
an end effector disposed at the distal portion of the shaft;
an actuator assembly disposed at the proximal portion of the shaft, the actuator assembly including a body and an actuator movable relative to the body;
a locking mechanism disposed on the body and configured to selectively lock the actuator in at least one configuration, the locking mechanism comprising a locking portion with a locking slot;
a biasing element disposed within the body and configured to bias the actuator proximally relative to the body, and
an actuation wire extending from the actuator assembly through the shaft to the end effector, wherein movement of the actuator relative to the body causes corresponding movement of the actuation wire to actuate the end effector, wherein a force exerted by the biasing element opposes actuation of the actuator.

17. The medical system of claim 16, wherein the locking mechanism further comprises a locking arm with a protrusion configured to engage with the locking slot.

18. The medical system of claim 17, wherein a first configuration of the medical system includes the protrusion positioned at a distalmost position of the locking portion, wherein the end effector is in a closed position in the first configuration, wherein the biasing element biases the protrusion against the distalmost position of the locking portion, wherein a second configuration of the medical system includes the protrusion positioned in the locking slot, wherein the end effector is in an open position in the second configuration, wherein the biasing element biases the protrusion against the distalmost position of the locking slot, wherein locking portion includes a first ramp and a second ramp, wherein the first ramp and the second ramp each include an angled portion and a straight portion, wherein the angled portions are angled relative to a central longitudinal axis of the medical instrument, wherein the straight portions are parallel to the central longitudinal axis of the medical instrument.

19. A medical instrument, comprising:
a shaft comprising a proximal portion and a distal portion;
an end effector disposed at the distal portion of the shaft;
an actuator assembly disposed at the proximal portion of the shaft, the actuator assembly comprising:
a body;
an actuator movable relative to the body; and
a locking mechanism, the locking mechanism comprising:
a first ramp disposed on the actuator and including a straight portion and an angled portion;
a second ramp disposed on the actuator and including a straight portion and an angled portion;
a locking slot disposed on the actuator between the first ramp and the second ramp; and
a locking arm pivotally coupled to the body via a pin, the locking arm including a protrusion configured to interact with the first ramp, the second ramp, or the locking slot upon actuation of the actuator; and
a biasing element disposed within the body and configured to bias the actuator proximally relative to the body; and
an actuation wire extending from the actuator assembly through the shaft to the end effector, wherein the biasing element applies a proximally-directed force to the actuator to maintain the end effector in a first configuration or a second configuration, wherein actuation of the actuator against the proximally-directed force of the biasing element causes the actuation wire to transition the end effector from the first configuration to the second configuration.

20. The medical instrument of claim 19, wherein the first ramp, the second ramp, and the locking slot form a chevron-shaped channel, wherein the protrusion of the locking arm translates through the channel as the actuator is actuated.
